(19) United States
(12) Patent Application Publication
(10) Pub. No.: US 2026/0096719 A1
(43) Pub. Date: Apr. 9, 2026

(54) CABLE-DRIVEN BENDING APPARATUS AND SYSTEM THEREOF

(71) Applicant: FLAT MEDICAL INC., TORTOLA (VG)

(72) Inventors: LI-YU LIN, Taipei City (TW); WEN-FU LUO, Taipei City (TW)

(21) Appl. No.: 19/351,421

(22) Filed: Oct. 7, 2025

Related U.S. Application Data
(60) Provisional application No. 63/704,032, filed on Oct. 7, 2024.

(51) Int. Cl. A61B 1/005

(52) U.S. Cl. CPC A61B 1/0052; A61B 1/0057

(57) ABSTRACT

A cable-driven bending apparatus is disclosed, configured for removable coupling with a control handle. The apparatus includes an elongated body with a deflectable distal end and a transmission unit at its proximal end. The transmission unit comprises a main shaft and a coaxially mounted rotatable ring. A first and second control wire extend from the transmission unit to the distal end; each coupled with the rotatable ring and wound around the shaft in opposite directions. Rotation of the ring selectively applies tension to one of the control wires, thereby deflecting the distal end. A system incorporating the cable-driven bending apparatus and a control handle is also disclosed. The control handle includes a housing, a controller, and a coupling and actuation mechanism, which together engage the transmission unit to control bending of the distal end. This configuration facilitates reliable manipulation, ease of assembly, and flexible replacement of the cable-driven bending apparatus.

### CROSS-REFERENCE TO RELATED APPLICATIONS

This application claims priority of U.S. Provisional Application No. 63/704,032, filed on Oct. 7, 2024, the entirety of which is incorporated by reference herein.

### FIELD OF THE INVENTION

The present invention relates to a cable-driven bending apparatus and system, particularly to a Bowden cable-based transmission mechanism for controlling the bending of a distal segment of an elongated body. Furthermore, the system includes a control handle and is capable of detachably connecting to one end of the cable-driven bending apparatus. The control handle is configured to drive the bending segment positioned at the distal end of the elongated body's insertion segment to perform bending operations.

### BACKGROUND OF THE INVENTION

Endoscopes are widely used medical devices for diagnosis and treatment. Although they differ in thickness, length, and internal channels based on their specific use, they generally follow similar principles. Usually, they comprise a front-end lens module, a tubular body, and a handle at the rear for operation and housing electronic components. More generally, bending control of elongated flexible instruments is commonly achieved through cable-driven mechanisms, such as Bowden cable systems, which utilize control wires arranged within an outer sheath to transmit force and enable precise distal bending.

A common use of endoscopy is assisted intubation, where the endoscope functions as a guide wire. For example, in bronchoscope-assisted endotracheal tube placement, the operator first slides the endotracheal tube over the outside of the bronchoscope. Then, the bronchoscope is advanced to the correct position. Because of its smaller diameter and adjustable bending at the tip, the bronchoscope makes insertion easier than the endotracheal tube alone. After confirming proper placement with the lens image, the bronchoscope acts as a guide wire, allowing the operator to push the pre-encased endotracheal tube into position to complete the intubation.

However, not all instances of pushing the tube after endoscope positioning are straightforward. In certain conditions, such as the presence of tumors, open wounds, inflammation, or other complicating factors, it may become quite challenging to push the tube smoothly and without causing additional trauma. These situations require careful technique and sometimes additional tools or interventions to ensure the tube is placed correctly and safely, minimizing patient discomfort and reducing the risk of complications. In such situations, the operator might need to change the tube size. However, since the tube is already encased in the endoscope and the handle is too bulky to withdraw the tube, a dilemma arises. The only options are: a) withdraw the endoscope from the patient, which requires restarting the entire procedure; or b) cut the endoscope, remove the handle, and extract the tube from the rear. Method a) takes much longer and can be risky, especially in urgent cases where every second counts, making it impractical. Although method b) is quicker, it is costly because it renders the endoscope unusable. Moreover, damaging the endoscope could pose additional risks, such as displacing the instrument or contaminating the surgical site with debris.

Although numerous related technologies and improvements have been disclosed over time, the primary challenge that remains in the development of separable endoscopes is the effective rejoining of control wires after they have been separated. This process is particularly complex due to the delicate nature of the control wires and the precision required for reconnection. Historically, many solutions attempted to address this issue by employing relatively complex and precise gear structures, which, while functional, pose significant limitations regarding practical application. These mechanical structures tend to be intricate and costly, potentially hindering their adoption, especially considering the current trend in endoscopic devices toward single-use, disposable instruments that aim to reduce costs and improve safety. Furthermore, the outer diameter of the endoscope must often be minimized to ensure compatibility with existing medical equipment, such as endotracheal tubes, which are essential for airway management during procedures. This constraint makes the design and implementation of separable endoscopes more challenging. Some endoscopes also require a working channel to facilitate suction, deliver therapeutic agents, or perform other patient treatments, which introduces additional restrictions on the scope's external size. Unfortunately, many previous separation designs have struggled to incorporate such working channels within the limited space available, often sacrificing functionality or increasing complexity. Overall, addressing these interconnected challenges—reliable wire reconnection, cost-effectiveness, size constraints, and multifunctionality—is crucial for advancing the practical use of separable endoscopes in clinical settings.

Therefore, the need for more versatile and user-friendly detachable endoscope systems remains unfulfilled, as current procedures can be inconvenient and complex. In summary, an improved detachable endoscope is urgently needed. Such a device would address the known drawbacks, offering easy assembly and operation while also reducing costs. More generally, the disclosed invention provides improvements to Bowden cable-based transmission systems, which may be advantageously applied not only to detachable endoscopes but also to other flexible instruments requiring controlled distal bending.

### SUMMARY OF THE INVENTION

The present invention relates to a novel cable-driven bending apparatus that addresses the limitations of prior art solutions by employing a rotation-based differential tension mechanism, in which a rotatable ring simultaneously drives two control wires in opposite directions—tensioning one while releasing the other—to achieve controlled distal bending. The core innovation of the invention lies in its force transmission mechanism, which enables easy detachment of an elongated tube from the handle and restoration of its functionality upon reassembly. Additionally, the invention ensures the inclusion of a working channel for suction and patient treatment without compromising the outer diameter.

In the first aspect, the present disclosure provides a cable-driven bending apparatus configured for removable coupling with a control handle to form a system, for example an endoscope system. The cable-driven bending apparatus includes an elongated body, a transmission unit, and first and second control wires. The elongated body has a deflectable distal end and a proximal end. The transmission unit connects to the proximal end of the elongated body and includes a first shaft that extends along the elongated body, as well as a rotatable ring mounted coaxially on the main shaft. Further, the first and second control wires are extended from the transmission unit and connect to the deflectable distal end respectively. Moreover, the first and second control wires are in contact with the rotatable ring and arranged to wrap around the first shaft in different directions. Additionally, the rotatable ring is configured to apply a tensioning force to either the first or second control wire by rotation.

In some embodiments, the cable-driven bending apparatus further includes a working channel having two openings, and the working channel extends through the elongated body and the transmission unit, wherein one opening is positioned at the deflectable distal end and the other opening is positioned at the proximal end of the transmission unit.

In some embodiments, the rotation of the rotatable ring simultaneously pulls one of the first or second control wires and releases the other, thereby causing the deflectable distal end to bend.

In some embodiments, the first and second control wires are secured to the rotatable ring.

In some embodiments, the fixed points of the first and second control wires on the rotatable ring are the same.

In some embodiments, the fixed points of the first and second control wires on the rotatable ring are different.

In some embodiments, the fixed points for the first and second control guide wires on the rotatable ring are symmetrically distributed relative to each other.

In some embodiments, when the transmission unit is in a normal stage, the tensioning force to the first and second control wires is the same, and the deflectable distal end is straight without any curvature.

In some embodiments, both first and second control wire sections on the main shaft are equal in length and the length satisfies the following equation:

$$L = \sqrt[2]{{H}^{2} + {( 2 π r )}^{2}} ,$$

wherein L represents the length of the first and second control wire sections on the first shaft, H represents a length of the first shaft, r represents an outer radius of the first shaft, and π represents the mathematical constant pi.

In some embodiments, when the transmission unit is in a rotation stage, the tensioning force to the first and second control wires is different, and the deflectable distal end is bent and points to one side.

In some embodiments, both first and second control wire sections on the first shaft are not equal in length, and a length of the longer section satisfies the following equation:

$$L + Δ l = \sqrt[2]{{H}^{2} + {( 2 π r )}^{2}} ,$$

wherein L represents an original length of the first and second control wire sections on the first shaft when the transmission unit is in a normal stage, Δl represents an increase in length of the first or second control wire sections on the first shaft, H represents a length of the first shaft, r represents an outer radius of the first shaft, and π represents the mathematical constant pi.

In some embodiments, the transmission unit further comprises a second shaft, and the rotatable ring is mounted between the first shaft and the second shaft.

In some embodiments, the first and second control wires are secured to the second shaft.

In some embodiments, the rotatable ring includes a groove configured to accommodate the first and second control wires, and when the rotatable ring rotates, the first and second control wires are actuated such that one of the wires is pulled while the other is released.

In some embodiments, the rotatable ring includes a channel configured to accommodate the first and second control wires, and when the rotatable ring rotates, the first and second control wires are actuated such that one of the wires is pulled while the other is released.

In some embodiments, when the transmission unit is in a rotation stage and the deflectable distal end reaches its maximum bending angle, one of the two control wires' sections on the first shaft has the shortest length.

In some embodiments, wherein an extension direction of one of the two control wires' sections on the first shaft having the shortest length is along a direction of the transmission unit.

In some embodiments, wherein the rotatable ring is rotated 180 degrees clockwise or 180 degrees counterclockwise.

In some embodiments, the elongated body includes a channel with an opening deposited at the proximal end of the elongated body, and the first and second control wires enter the channel through the opening and connect to the deflectable distal end.

In some embodiments, the elongated body includes at least two channels and each of the channels has an opening deposited at the proximal end of the elongated body, and the first and second control wires each enter one of the channels through one of the openings and connect to the deflectable distal end.

In some embodiments, the two openings at the proximal end of the elongated body are symmetrically distributed relative to each other.

In the second aspect, the present disclosure also provides another cable-driven bending apparatus configured for removable coupling with a control handle. The cable-driven bending apparatus includes an elongated body, a transmission unit, and first and second control wires. The elongated body has a deflectable distal end and a proximal end. The transmission unit is positioned at a proximal end of the elongated body, and it includes first and second shafts extending along with the elongated body and a rotatable ring mounted coaxially between the first shaft and the second shaft. The first and second control wires are extended from the transmission unit and connect to the deflectable distal end respectively. The other ends of the first and second control wires connect to the first shaft, and a portion of the first and second control wires are in contact with the rotatable ring and arranged to wrap around the first and second shafts in different directions. Furthermore, the rotatable ring is configured to apply a tensioning force on either the first or second control wire by rotation.

In some embodiments, when the transmission unit is in a normal stage, the tensioning force on the first and second wires is the same, and the deflectable distal end is straight without any curvature.

In some embodiments, when the transmission unit is in a rotation stage, the tensioning force on the first and second wires is different, and the deflectable distal end is bent and points to one side.

In the third aspect, the present disclosure further provides a system, which includes the cable-driven bending apparatus as previously mentioned and a control handle. Further, the control handle includes a housing, a controller installed in the housing, and a coupling and actuation mechanism configured within the housing. Furthermore, the detachable scope is detachably connected with the control handle, and the controller drives and controls the transmission unit of the cable-driven bending apparatus via the coupling and drive mechanisms, thereby causing the deflectable distal end of the elongated body to bend.

In some embodiments, the cable-driven bending apparatus includes a working channel having two openings, and the working channel extends through the elongated body and the transmission unit, wherein one opening is positioned at the deflectable distal end and the other opening is positioned at the proximal end of the transmission unit.

In some embodiments, the control handle comprises a working channel port detachably connected to the working channel.

This coupling mechanism, along with its corresponding design features, not only simplifies endoscope-assisted intubation procedures but also enhances the overall versatility and functionality of the detachable endoscope system. Moreover, the ease of manufacturing and cost-effectiveness of this design make it a practical solution for medical professionals. By offering a simplified yet efficient method for separating and rejoining endoscope components, this invention aims to improve patient care outcomes while minimizing procedural complexities. In conclusion, the present invention represents a significant advancement in detachable endoscope technology, promising greater ease of use, enhanced functionality, and improved patient outcomes in medical settings.

### BRIEF DESCRIPTION OF DRAWINGS

One or more embodiments are illustrated by way of example, and not by limitation, in the figures of the accompanying drawings, wherein elements having the same reference numeral designations represent like elements throughout. The drawings are not to scale, unless otherwise disclosed. Certain parts of the drawings are exaggerated for explanation purposes and shall not be considered limiting unless otherwise specified.

FIG. 1 illustrates an exemplary system having a cable-driven bending apparatus and a control handle.

FIG. 2 illustrates a disassembly diagram of the system, indicating that the cable-driven bending apparatus and the control handle are detachable.

FIG. 3 illustrates the transmission unit with the X-shape steel wire configuration.

FIG. 4 illustrates a front view and a side cross-section of the transmission unit and the tubular portion of the detachable scope, and also includes a cross-sectional view at the junction where the transmission unit connects to the tubular portion.

FIG. 5 illustrates a side view and a front sectional view of the transmission unit along with a portion of the tubular portion of the detachable scope.

FIGS. 6 and 7 illustrate the effects of clockwise and counterclockwise rotation of the rotatable ring on the bending of the tubular tip.

FIG. 8 illustrates an overview of the control transmission from the handle to the transmission unit, and an exemplary toothed gear structure inside the handle.

FIGS. 9(A) and (B) show a simplified schematic of how control wires are arranged on the main shaft surface during the “Normal Stage” of the transmission unit.

FIG. 10 shows the relationship between the outer radii of the main shaft and the control of the guiding's pulling distance wires.

FIG. 11 shows the relationship between the main shaft height and the selected radius of the main component shaft.

FIG. 12 illustrates the structural design of the transmission unit and a portion of the tubular body in the “Normal State”, where (A) shows a front view and (B) shows a rear view.

FIG. 13 illustrates the structural design of the transmission unit and a portion of the tubular body in the “Rotation State”, where (A) shows a front view and (B) shows a rear view.

FIG. 14 illustrates another specific embodiment of the detachable scope and its transmission unit design, where the transmission unit is in the “Normal Stage.”

FIGS. 15 and 16 illustrate a particular embodiment of the present detachable scope and its transmission unit, wherein said transmission unit is located within a “Rotation Stage.”

The drawings provided are purely schematic representations and should not be viewed as limiting or definitive. Within these drawings, some of the elements' sizes may be intentionally exaggerated or not to scale, serving illustrative purposes rather than precise measurements. The stated dimensions, as well as the relative sizes and proportions depicted, do not necessarily reflect the actual reduced scale or real-world implementation of the invention or device in question. Furthermore, any reference signs or labels included in the claims should not be interpreted as limiting the scope of the invention or the claims themselves. Similar reference symbols used across various drawings are intended to indicate like or corresponding elements, components, or features, thereby aiding in understanding the drawings and the invention's overall concept. These clarifications are meant to ensure that the drawings are understood correctly in the context of the description and claim scope, avoiding any misinterpretation that might arise from their schematic nature or illustrative exaggerations.

### DETAILED DESCRIPTION OF THE INVENTION

Unless defined otherwise, all technical and scientific terms used herein have the same meanings as commonly understood by one of skill in the art to which this disclosure belongs. It will be further understood that terms; such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In the drawings, like reference numbers are used to designate like or similar elements throughout the various views, and illustrative embodiments of the present disclosure are shown and described. The figures are not necessarily drawn to scale, and in some instances the drawings have been exaggerated and/or simplified in places for illustrative purposes. One of ordinary skill in the art will appreciate the many possible applications and variations of the present disclosure based on the following illustrative embodiments of the present disclosure.

It will be understood that when an element is referred to as being “on” another element, it can be directly on the other element or intervening elements may be present. In contrast, when an element is referred to as being “directly on” another element, there are no intervening elements present.

It will be understood that singular forms “a”, “an” and “the” are intended to include the plural forms as well, unless the context clearly indicates otherwise. Furthermore, relative terms, such as “bottom” and “top,” may be used herein to describe one element's relationship to other elements as illustrated in the Figures.

It will be understood that elements described as “under” or “below” other elements would then be oriented “over” or “above” the other elements. The exemplary terms “under” or “below” can, therefore, encompass both an orientation of over and under.

Unless otherwise defined, all terms (including technical and scientific terms) used herein have the same meaning as commonly understood by one of ordinary skill in the art to which this disclosure belongs. It will be further understood that terms; such as those defined in commonly used dictionaries, should be interpreted as having a meaning that is consistent with their meaning in the context of the relevant art and the present disclosure, and will not be interpreted in an idealized or overly formal sense unless expressly so defined herein.

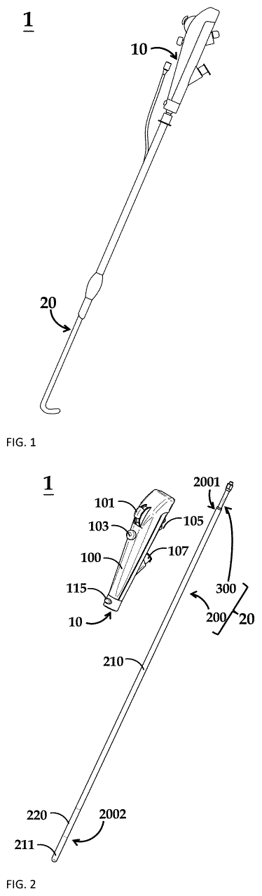

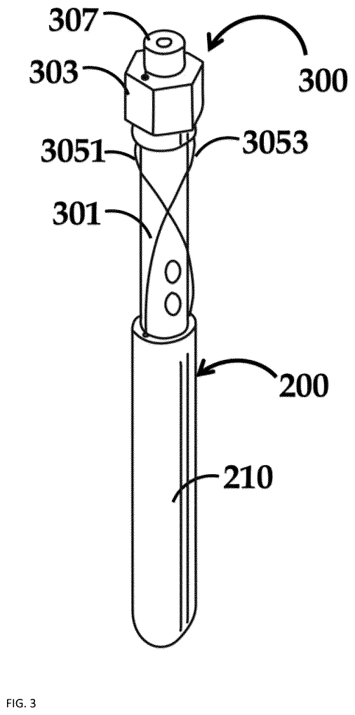

FIGS. 1 and 2 provide an overview of the overall appearance and structural features of the detachable system that is disclosed herein. These figures serve to illustrate the detailed design and functional aspects of the system. Please refer to FIG. 1, the detachable system 1 includes a control handle 10 and a cable-driven bending apparatus 20. Furthermore, the cable-driven bending apparatus 20 includes a tubular portion 200 and a transmission unit 300. The transmission unit 300 is designed to connect with the tubular portion 200 and detachably attach to the control handle 10, facilitating easy assembly and disassembly. Please refer to FIG. 2, the control handle 10 of the detachable endoscope system 1 serves as the main interface for the operator to control the detachable endoscope system 1. In addition to a grip 100 that allows for single-handed operation, the control handle 10 may include features such as a bending control lever 101, a suction button 103, a suction connector 105, and a working channel port 107. Furthermore, one end of grip 100 is equipped with socket 115. Notably, the shape and structural design of the grip 100 can incorporate ergonomic principles to ensure a comfortable hold and ease of manipulation during medical procedures. Furthermore, additional interfaces not mentioned above can be added as needed. Another aspect, the cable-driven bending apparatus 20 includes a tubular portion 200 and a transmission unit 300. Furthermore, the tubular portion 200 mainly consists of a tubular body 210 and a flexible portion 220 positioned near the tubular tip 211. In other words, the tubular tip 211 and a flexible portion 220 form a deflectable portion at the distal end of the tubular body 210.

Most importantly, as mentioned earlier, the control handle 10 and the detachable scope 20 are connected in a detachable manner, which allows for greater flexibility and ease of maintenance. More specifically, the grip 100 and the transmission unit 300 are connected in a detachable manner, thereby enabling the system to be conveniently assembled and disassembled without requiring complex tools or procedures. This detachable connection significantly enhances the detachable endoscope system's versatility and adaptability in various clinical settings, allowing for quick and efficient interchangeability of components, which can be particularly beneficial during urgent procedures or when modifications are needed for different diagnostic or treatment scenarios. Overall, this design feature contributes to improved usability, maintenance efficiency, and cost-effectiveness of the system. For example, earlier, when a clinician needs to temporarily adjust the size or diameter of a tube during a patient's treatment, such as in cases involving tumors, open wounds, inflammation, or other complex conditions, they only need to detach grip 100 from the transmission unit 300 on the detachable endoscope system 1. At this stage, the cable-driven bending apparatus 20 remains accurately positioned where the clinician wants it. The ill-fitting catheter can be removed and replaced with a suitable one, then smoothly advanced along the cable-driven bending apparatus 20 back into the patient's body to reach the target site. The clinician can then reconnect grip 100 to transmission unit 300 and resume using the detachable endoscope system 1 to guide the catheterization, minimizing additional trauma, reducing patient discomfort, and lowering risk complications.

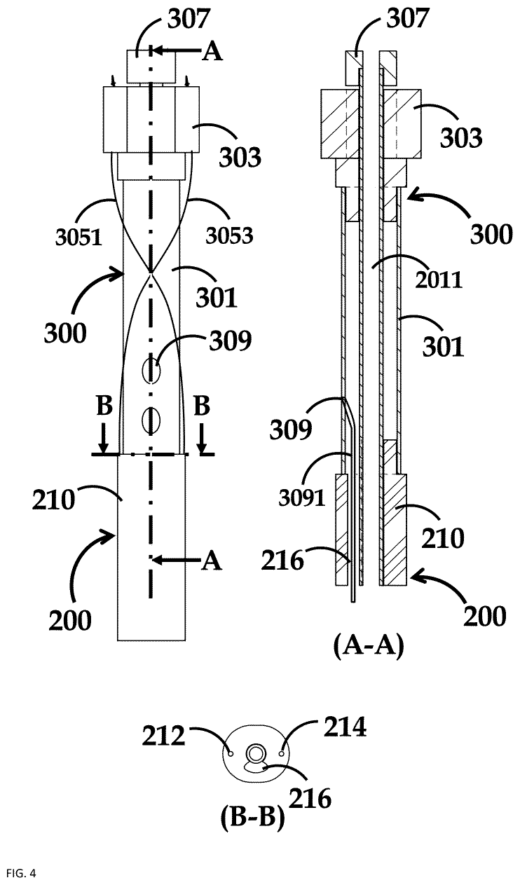

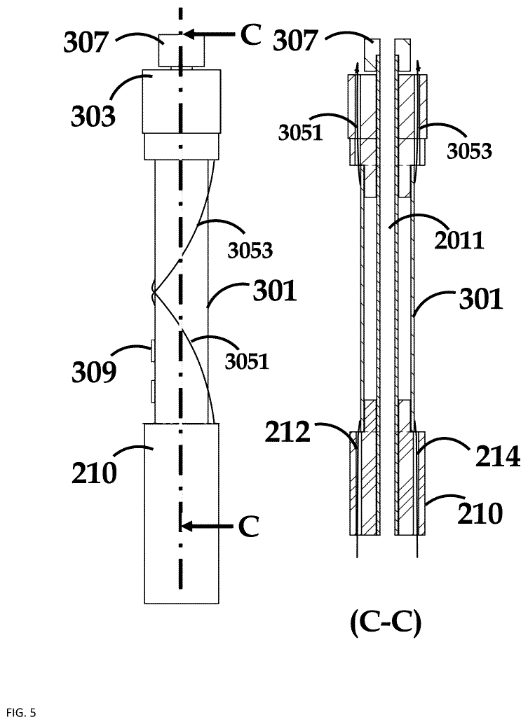

Please refer to FIG. 3. The transmission unit 300 is a vital component responsible for transmitting the operator's manipulation force to control the degree of bending of the flexible portion 220 on the tubular portion 200, thereby further controlling the pointing direction of the tubular tip 211. The tubular portion 200 has an elongated body, and it also has a distal end 2002 and a proximal end 2001. Furthermore, the transmission unit 300 is connected to the proximal end 2001 of the tubular body 210. In this example, the transmission unit 300 mainly consists of the main shaft 301, rotatable ring 303, control wire 305, and working channel connector 307. Additionally, the rotatable ring 303 connects to the proximal end of the main shaft 301, and the rotatable ring 303 is coaxially arranged with the main shaft 301, enabling both components to rotate in a coaxial manner. The control wire 305 includes two control wires, namely the first and second control wires 3051 and 3053. One end of both first and second control wires 3051 and 3053 is respectively connected to rotatable ring 303, while the other end of control wires 3051 and 3053 is respectively threaded into tubular body 210 and attached to tubular tip 211. In other words, each control wire has one fixed point on the rotatable ring 303, and these two fixed points are symmetrically arranged relative to each other rotatable (see FIG. 4). Furthermore, the first and second control wires 3051 and 3053 extend from tubular body 210 to form an X-shaped configuration (i.e., the two wires intersect each other) on the main shaft 301 of the transmission section 300. In this configuration, control wires 3051 and 3053 are under no tension, and tubular tip 211 of tubular portion 200 stays upright, forming the system's “Normal Stage”. In a preferred embodiment, the control wire may be made of materials that exhibit high strength, toughness, and resistance to high temperatures, such as steel or similar metallic or non-metallic materials.

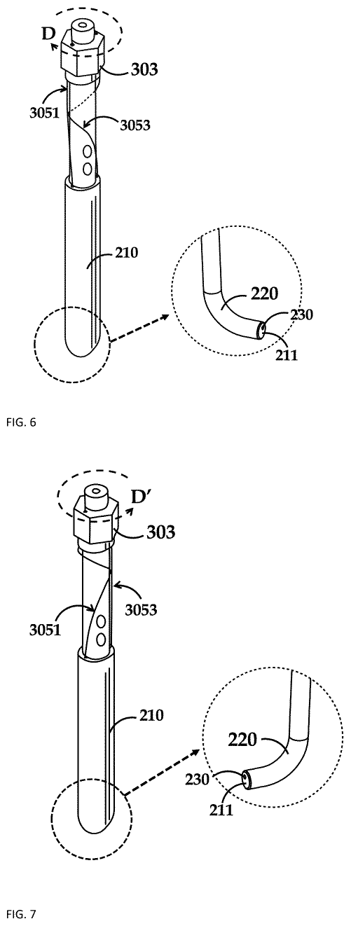

FIG. 4 illustrates a front view and a side cross-section of the transmission unit 300, along with a section of the tubular portion 200 of the cable-driven bending apparatus 20. It also includes a cross-sectional view at the junction where the transmission unit 300 connects to the tubular portion 200. FIG. 5 illustrates a side view and a front sectional view of the transmission unit 300 along with a portion of the tubular portion 200 of the detachable scope 20. As FIGS. 4 and 5 disclose, the transmission unit 300 of the detachable scope 20 is in the normal state, and both figures highlight the connection configuration of the control wires 3051 and 3053 that connect to the rotatable ring 303. Specifically, FIGS. 4 and 5 both illustrate the control wires 3051 and 3053, respectively, extending from channels 212 and 214 of the tubular body 210 and separately connecting to the rotatable ring 303. Notably, in this embodiment, the parts of control wires 3051 and 3053 that extend beyond channels 212 and 214 (i.e., the sections on the surface of the main shaft 301 of the transmission unit 300) are arranged in an X-shaped pattern through interlacing with each other. Furthermore, as FIG. 4 shows, another direction of the sectional view reveals the camera's wires 3091 extending from a first groove 216 of the tubular body 210 and connecting to the electrical contacts 309 on the main shaft 301. Additionally, inside the detachable scope 20, a working channel 2011 runs through the transmission section 300 and the tubular section 200, and it is used for suction and permits instrument passage.

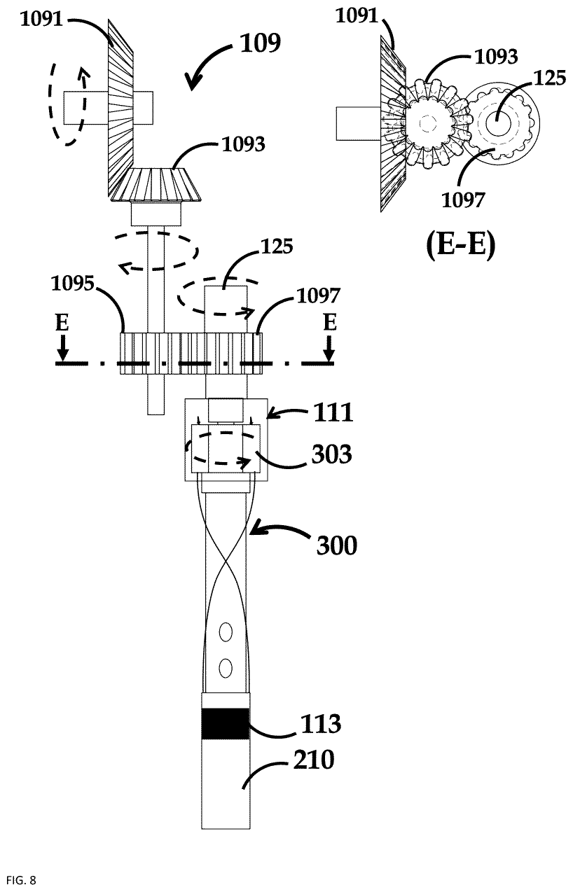

FIGS. 6 and 7 illustrate the results of rotatable the ring 303 by 180 degrees from the “normal state” to the “rotated state,” with FIG. 6 depicting a clockwise rotation and FIG. 7 showing a counterclockwise rotation. Refer to FIG. 6, which illustrates how rotatable the rotatable ring 303 causes the flexible section 220 to bend, making the tubular tip 211 point to the right. Specifically, when an operator rotates the rotatable ring 303 clockwise (i.e., the direction D), part of the control wire 3053 that was originally inside channel 214 of the tubular body 210 is pulled out, while part of the control wire 3051 that was outside channel 212 of the tubular body 210 is guided into it. In other words, turning the rotatable ring 303 clockwise (i.e., the direction D) decreases the length of control wire 3053 in channel 214 of the tubular body 210, while increasing the length of control wire 3051 in channel 212 of the tubular body 210. This action causes the flexible portion 220 to bend to the right, directing the tubular tip 211 toward the right. Conversely, refer to FIG. 7, which illustrates how rotatable the rotatable ring 303 causes the flexible section 220 to bend, making the tubular tip 211 point to the left. Specifically, similar to the previous mention, when an operator rotates the rotatable ring 303 counterclockwise (i.e., the direction D′), part of the control wire 3051 that was originally inside channel 212 of the tubular body 210 is pulled out, while part of the second control wire 3053 that was outside channel 214 of the tubular body 210 is guided into it. In other words, turning the rotatable ring 303 counterclockwise (i.e., the direction D′) decreases the length of the first control wire 3051 in channel 212 of the tubular body 210, while increasing the length of the second control wire 3053 in channel 214 of the tubular body 210. This action causes the flexible portion 220 to bend to the left, directing the tubular tip 211 in that direction.

The current design enables precise adjustment of the flexible portion 220's bend angle, directly guiding the pointing or advancing direction of the tubular tip 211. When the rotatable ring 303 turns, it pulls one control wire, 3051 or 3053, while releasing tension on the other, allowing smooth and accurate endoscope operation during medical procedures. In other embodiments, the tubular tip 211 is equipped with a camera 230 that captures images, helping to verify the position of the tubular tip 211. Moreover, the first and second control wires 3051 and 3053, which extend from the tubular body 210, are arranged crosswise inside the rotatable ring 303, creating an X-shape. This setup ensures precise control of the flexible portion 220's bending when the rotatable ring 303 rotates.

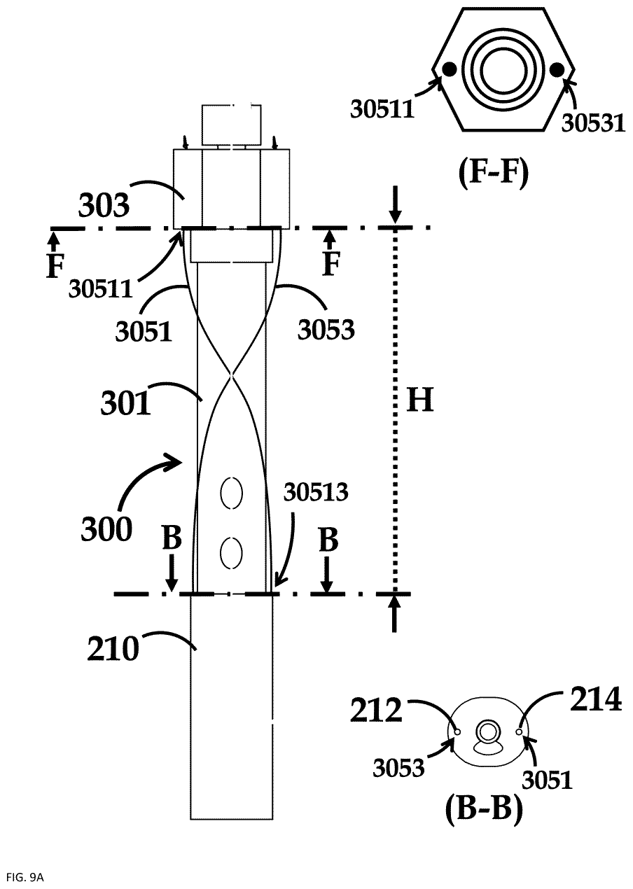

FIG. 8 illustrates that a coupling and actuation mechanism 109 is installed within the control handle 10 to connect and transfer motion to the transmission unit 300. Specifically, in the present embodiment, the coupling and actuation mechanism 109 is a mechanical structure made up of a plurality of gear sets. However, in other embodiments, the coupling and actuation mechanism 109 does not have to consist of gear sets; it could also be directly made with electronic components combined with a motor. In other words, the suitable mechanism can be designed differently depending on specific requirements. The control handle 10 contains a coupler 111, which is structured to couple with the rotatable ring 303. Therefore, when the transmission unit 300 is inserted into the handle 100, the rotatable ring 303 on the transmission unit 300 couples with the coupler 111 inside the handle 100. As a result, the bending control lever 101 can further operate the coupling and actuation mechanism 109 to rotate the rotatable ring 303 in either a clockwise or counterclockwise direction. Additionally, the side wall of tubular body 210 is equipped with plug 113, which is structurally designed to form a detachable connection with socket 115 on handle 100. As previously mentioned, in the present embodiment, the coupling and actuation mechanism 109 installed within the handle 100 is a mechanical structure composed of multiple gear sets 1091, 1093, 1095, and 1097 and other suitable means for the transmission of force. These gears are strategically positioned within the handle 100 to effectively transfer force resulting from the operator's manipulation. Additionally, the gear reduction ratio is adjustable to optimize the force transmission process, thereby reducing the exertion required by the operator. The coupler 111, as described above, is equipped with a channel and is connected to tube 125, facilitating an airtight connection between the working channel port 107 of the control handle 10 and the working channel 2011 of the detachable scope 20. Moreover, due to the smaller outer diameter of the working channel port 107 of the control handle 10 compared to tube 125, interference that may impact force transmission is minimized.

The present power transmission setup (i.e., coupling and actuation mechanism 109) boosts the overall performance and usability of the endoscopic system through a dependable and efficient force transfer method. Additionally, the ability to adjust the gear reduction ratio enhances the user experience by making operation easier. This novel design represents substantial progress in endoscopic device technology, offering improved performance and a more satisfying user experience.

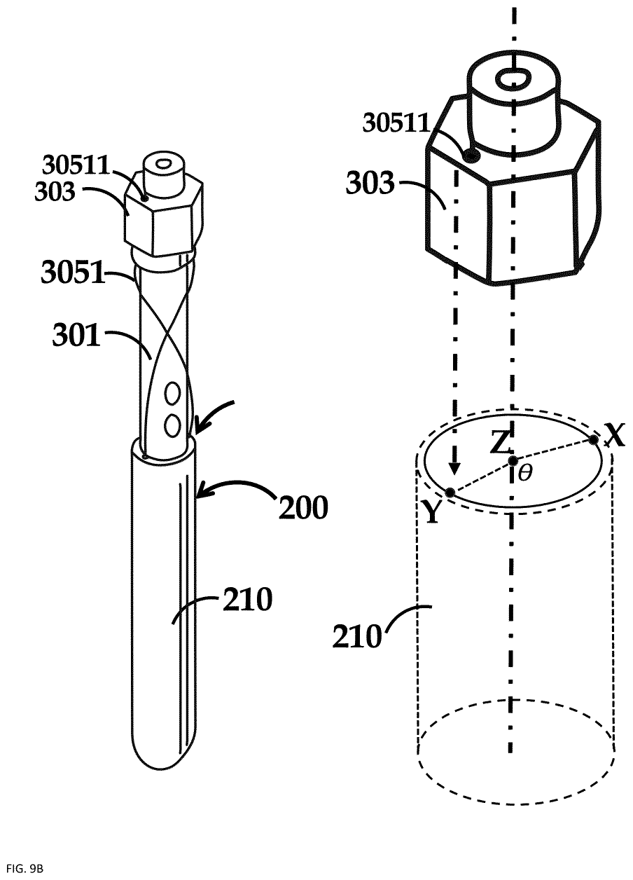

As previously mentioned, a key element of the disclosed setup is the design of transmission unit 300. Specifically, how control wires 3051 and 3053 are arranged on the surface directly affects the bending of the flexible section 220. FIG. 9A illustrates a simplified schematic of the distribution of the first and second control wires 3051 and 3053 on the main shaft 301 surface during the “Normal Stage” of transmission unit 300. Section (F-F) depicts a cross-section at the junction between rotatable ring 303 and main shaft 301; in section (B-B), a cross-section at the junction between main shaft 301 and tubular body 210. As shown in the sectional view (F-F), control wires 3051 and 3053 are symmetrically arranged at their fixing points (30511 and 30531) on rotatable ring 303. The channels 212 and 214 on the tubular body 210, which house the first and second control wires 3051 and 3053, respectively, and the openings 30513 and 30533 of the channels 212 and 214 are also symmetrically arranged. Furthermore, assuming the distance H between the bottom surface (i.e., the distal surface that adjacent to main shaft 301) of rotatable ring 303 and the top surface (i.e., the proximal surface that adjacent to main shaft 301) of tubular body 210, the outer radius r of main shaft 301, and the minimum length of control wire 3051 or 3053 that is exposed on the main shaft 301 (meaning the part not inside channels 212 and 214) is L_{NS}, then the relationship between H, r, and L_{NS} satisfies the following Equation 1.

$${L}_{NS} = \sqrt[2]{{H}^{2} + {( \frac{{θ}_{NS}}{180 °} \times π r )}^{2}} \qquad Equation 1$$

Additionally, π represents the mathematical constant pi. Please refer to FIG. 9B. In this embodiment, if the projections (i.e., points Y and X) of the fixing point 30511 and the opening 30513 are superimposed onto the same plane, and the intersection point of the coaxial axis of the main shaft 301, rotatable ring 303, and tubular body 210 with this plane is considered the center (i.e., point Z), then drawing a circle with the outer radius (i.e., the r) of the main shaft 301 will cause the circle's circumference to precisely pass through points X and Y. θ_{NS} represents the angular difference between points X and Y on the circumference. In other words, θ_{NS} represents an angular difference between two ends of either control wire on the upper portion of the first shaft. Specifically, θ_{NS} represent the angle subtended by the center of the sector formed by Points XYZ in the “Normal Stage”. In the present embodiment, the angle θ is 180 degrees. Therefore, the length L_{NS} of the first or second control wire should be

$${L}_{NS} = \sqrt[2]{{H}^{2} + {( π r )}^{2}} .$$

In a preferred embodiment, the value of θ_{NS} is 0°<θ_{NS}≤180°.

Furthermore, when the user operates the bending control lever 101 to drive the coupling and actuation mechanism 109, rotatable the rotatable ring 303 causes the transmission unit 300 to transition from “Normal Stage” to “Rotation Stage” (as illustrated in FIG. 6 or 7). As described above, portions of control wires 3051 and 3053 originally located within channels 212 and 214 are withdrawn. Assuming the length of the withdrawn portions of control wires 3051 and 3053 is Δl, then the total length of the control wire 3051 or 3053 that is exposed on the main shaft 301 (meaning the part not inside channels 212 and 214) is L_{RS} (i.e., L_{NS}+Δl). In the present embodiment, when the rotatable ring 303 is turned to its maximum limit angle (i.e., rotated 180 degrees) to attain the greatest bending of the flexible portion 220, one of the control wires 3051 and 3053 on the surface of the main shaft 301 has the shortest length, while the other has a relatively longer length. For example, see FIGS. 7 and 8 simultaneously. When the rotatable ring 303 is driven to rotate counterclockwise, causing the flexible section 220 to bend leftward, the part of the control wire 3051 exposed on the surface of the main shaft 301 is in its shortest state. This is because the connection point 30511 of the control wire 3051 on the rotatable ring 303 has the shortest distance to the opening of the channel 214. Conversely, the length (i.e., L_{RS}; the length in the “Rotation Stage”) of the portion of the control wire 3053 exposed on the surface of the main shaft 301 at this point should be L_{NS}+Δl. The relationship between H, r, Δθ and L+Δl satisfies the following Equation 2. Furthermore, π represents the mathematical constant pi, and Δθ represents the rotation angle of the rotatable ring 303.

$${L}_{RS} = {L}_{NS} + Δ l = \sqrt[2]{{H}^{2} + {( \frac{{θ}_{NS} + Δθ}{180 °} \times π r )}^{2}} \qquad Equation 2$$

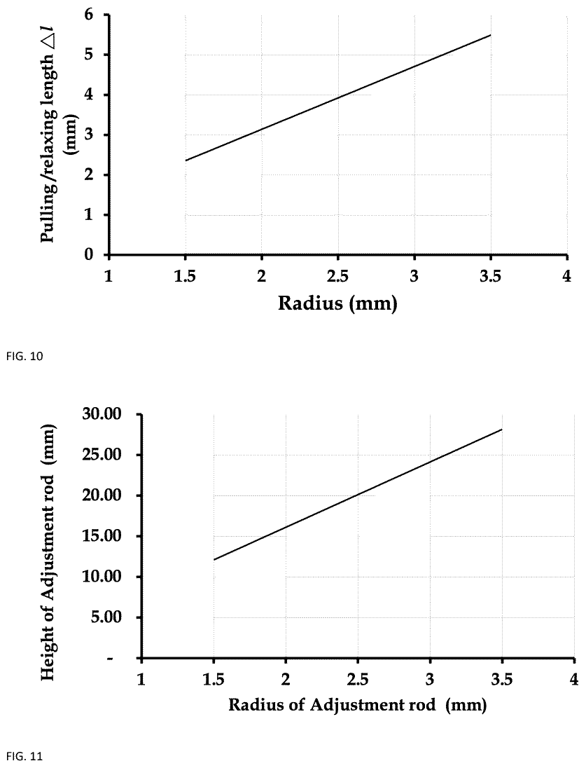

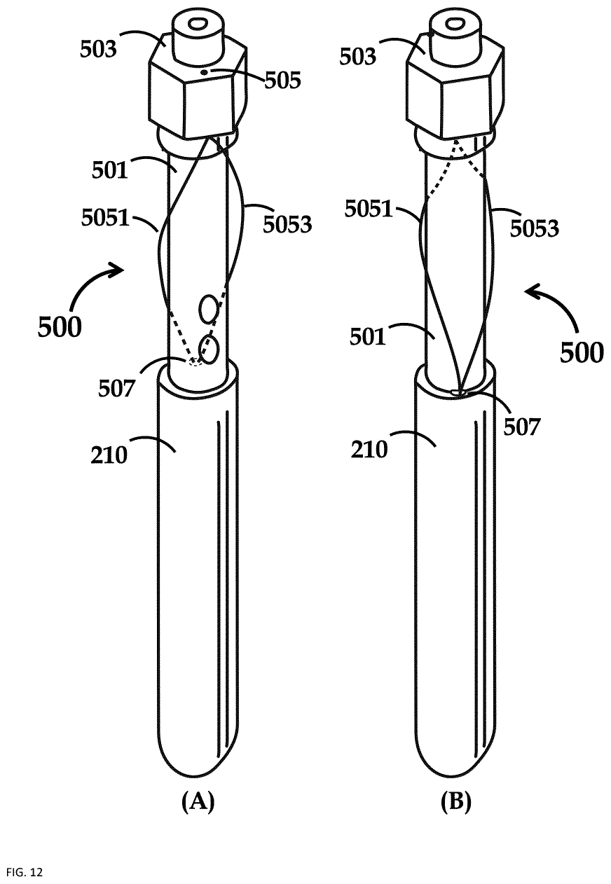

FIG. 10 shows the relationship between different outer radii of the main shaft 301 and the corresponding control of the pulling distance of control wires 3051 and 3053. When the outer diameter of the tubular body 210 is 3 mm (radius of 1.5 mm), the control wires 3051 and 3053 on each side need to be pulled approximately 2.4 mm and relaxed by about 2.4 mm, respectively, to achieve a 90-degree bend in the flexible portion 220. This length remains unaffected by the curvature of the bend but depends on the outer diameter of the tubular body 210. Therefore, when the outer diameters of the tubular body 210 are 3 mm, 4 mm, 5 mm, 6 mm, and 7 mm, the pulling and relaxing distances for control wires 3051 and 3053 are 2.4 mm, 3.1 mm, 3.9 mm, 4.7 mm, and 5.5 mm, respectively. Furthermore, FIG. 11 shows the relationship between the height of the main shaft 301 (as mentioned earlier in FIG. 9) and the chosen radius of the main shaft 301, based on the data provided earlier and in FIG. 9. Specifically, when the outer diameters of the transmission unit's body are 3 mm, 4 mm, 5 mm, 6 mm, and 7 mm, the minimum height of the main shaft 301 in the transmission unit 300 are 12.1 mm, 16.1 mm, 20.1 mm, 24.1 mm, or 28.2 mm respectively. This highlights the design's feasibility, especially in reducing the overall size of the structure when working with smaller outer diameters.

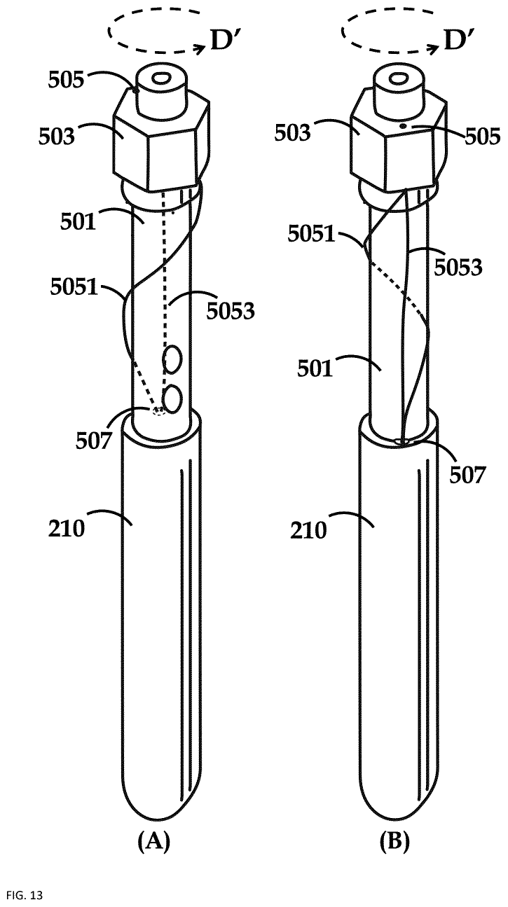

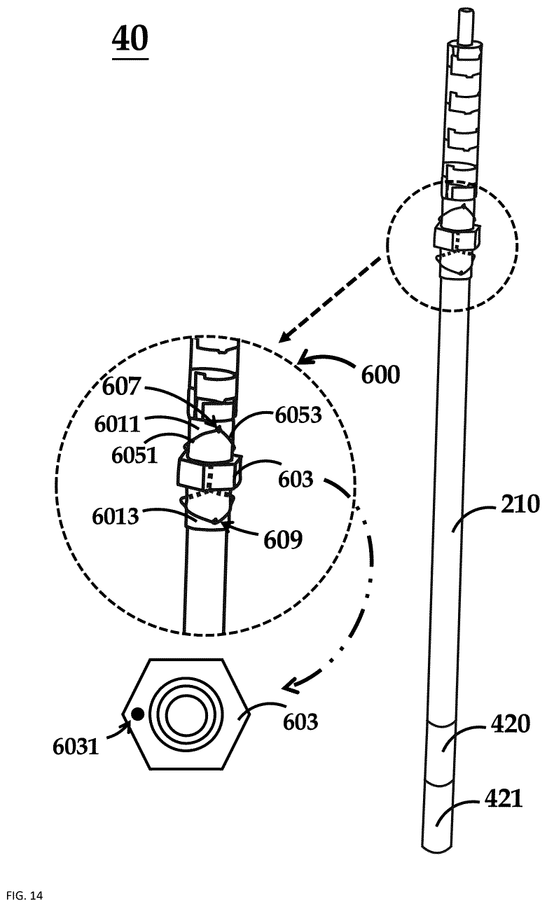

The present disclosure also provides another embodiment (see FIGS. 12 and 13) of the transmission unit 500. Specifically, the difference between the transmission unit 500 in this embodiment and the transmission unit 300 in the previous embodiment lies in the configuration of the first and second control wires and their respective fixing points. Please refer to FIG. 12, which illustrates the structural design of transmission unit 500 and a portion of tubular body 210 in the normal state, where (A) shows a front view and (B) shows a rear view. Similar to the transmission unit 300, the transmission unit 500 connects to the proximal end of the tubular body 210 and includes a rotatable ring 503 and a main shaft 501. One end of the main shaft 501 connects to the proximal end of the tubular body 210, while the other end connects to the rotatable ring 503. Notably, one end of both the first and second control wires 5051 and 5053 is secured to the same fixed point 505, and sections of these control wires on the main shaft 501 are arranged to wrap around the shaft in different directions. Moreover, as shown in the figure, neither the first nor the second control wires 5051 and 5053 cross each other, unlike in the previous embodiment. Additionally, both the first and second control wires 5051 and 5053 enter the channel (not shown) inside the tubular body 210 through the same opening 507 and are further connected to the deflectable distal end, including the flexible section 220 and the tubular tip 211, of the tubular body 210 and enter the channel (figure not shown) within the tubular body 210 through the same opening 507 and are further connected to the deflectable distal end (the flexible section 220 and the tubular tip 211) of the tubular body 210.

Please refer to FIG. 13, which illustrates the structural design of transmission unit 500 and a portion of tubular body 210 in the rotation state, where (A) shows a front view and (B) shows a rear view. Notably, in FIG. 13, the rotatable ring 503 is rotated 180 degrees counterclockwise. Therefore, as described in the previous embodiments, in this state (i.e., the rotation stage), the first control wire 5051 is pulled taut by the force imparted by the rotation of the rotatable ring 503, while the second control wire is correspondingly slackened. Further, Furthermore, as shown in the figure, the fixed point 505 on the rotatable ring 503 is now at its closest distance to the opening 507. Consequently, the portion of the second control wire 5053 on the main shaft 501 achieves its shortest possible length (i.e., it assumes a straight-line configuration). However, another notable aspect of this embodiment is that neither the first nor the second control wires will cross each other as in the previous embodiment, whether in the normal stage or during the rotation stage. The non-crossing configuration of the first and second control wires, as described in the present embodiment, offers several advantages over the previous embodiment. Because the two control wires are arranged on separate paths without intersecting each other, the tension applied during bending is transmitted more uniformly and efficiently along the main shaft. This not only minimizes frictional interference between the wires but also enhances the precision and smoothness of the bending response. Furthermore, the absence of wire crossing reduces the risk of mechanical wear, deformation, or entanglement during repeated actuation cycles, thereby improving the overall structural stability and durability of the transmission unit. Consequently, this configuration provides more consistent force feedback and improved controllability, making it particularly suitable for compact or disposable cable-driven devices where operational reliability and manufacturing simplicity are critical.

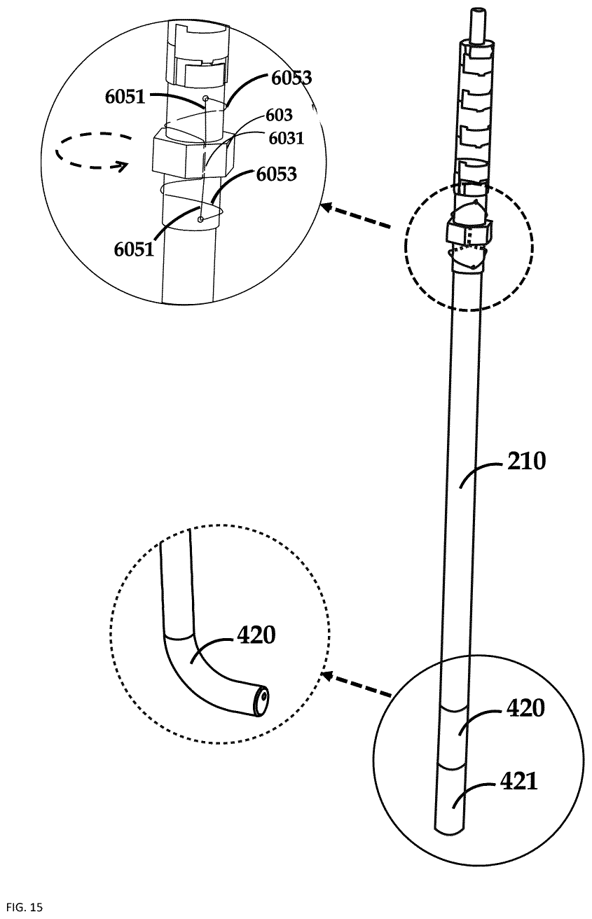

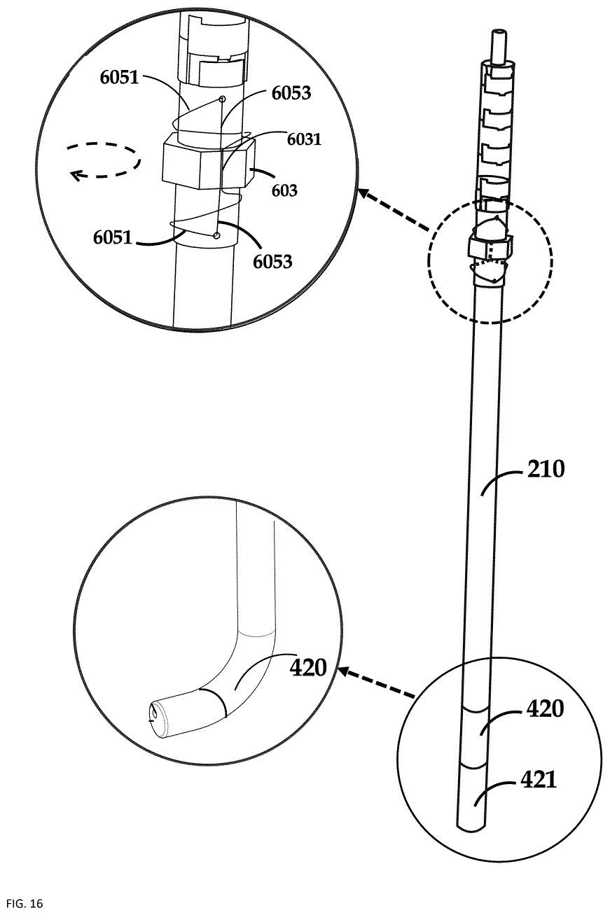

This disclosure further provides another specific embodiment (see FIGS. 14, 15, and 16) of a detachable scope (i.e., detachable scope 40), which can also be used in conjunction with the control handle 10 of the aforementioned endoscope system 1. That is, the control handle 10 can also control the detachable scope 40. More specifically, the detachable scope 40 is mainly designed for endoscopes with a larger diameter in the tubular body and main shaft. Please refer to FIGS. 14 and 15, the bending of the flexible portion 420 is directly related to the diameter of the tubular portion, which, in turn, affects the pulling and relaxing length of the control wires (6051 or 6053). The transmission unit 600 controls the tension of the control wires 6051 and 6053, and this control is further influenced by the diameter of the first shaft 6011 and the second shaft 6013. Therefore, for scopes with larger diameters or in cases where a bending angle greater than 90 degrees is required, the previously described embodiments may not suffice.

This embodiment introduces a rotatable knob 603 located between the first shaft 6011 and the second shaft 6013. The ends of the two control wires 6051 and 6053 are both fixed at a point 607 at the proximal end of the first shaft 6011. Furthermore, both control wires, 6051 and 6053, are then in contact with a groove 6031 on the rotatable knob 603 and proceed through an opening 609 at the distal end of the second shaft 6013. In another embodiment, the rotatable knob 603 includes a channel (figure not shown), and both control wires 6051 and 6053 then pass through the channel on the rotatable knob 603 and proceed through an opening 609 at the distal end of the second shaft 6013. Please refer to FIGS. 15 and 16, when the rotatable knob 603 is rotated, one of the control wires (6051 or 6053) is released while the other (6053 or 6051) is pulled.

The primary difference between this specific embodiment and the preceding one lies in the rotatable knob 603 being positioned at the center of the adjustment rod, i.e., between first shaft 6011 and the second shaft 6013. Additionally, the fixation and traction designs of control wires 6051 and 6053 have been modified. Consequently, this embodiment effectively doubles the length of wire extension and retraction, enabling smoother and more precise operation. Furthermore, the absence of interference between the two wires during operation ensures reliable performance. As depicted in FIG. 15, the rotatable knob 603 is shown rotatable counterclockwise by 180 degrees. This rotation causes the control wires 6051 to release tension while simultaneously tightening the control wires 6053. This coordinated movement is crucial for adjusting the bending mechanism of the device. Similarly, in FIG. 16, the rotatable knob 603 rotates clockwise by 180 degrees, resulting in the control wires 6053 releasing tension and the control wires 6051 tightening. This reversible action allows for precise control over the bending process. The design of this mechanism ensures smooth and accurate manipulation of the flexible portion 420, enabling effective bending even at larger angles or when used with scopes that have larger tubular diameters. The ability to finely tune the tension in the control wires enhances the scope's maneuverability, providing the operator with better control during procedures. Overall, this feature contributes significantly to the functionality and versatility of the bending control system, making it well-suited for a variety of medical and technical applications.

1. A cable-driven bending apparatus, configured for removable coupling with a control handle, comprising:
an elongated body having a deflectable distal end and a proximal end;
a transmission unit connecting to the proximal end of the elongated body, comprising:
a first shaft extending along with the elongated body; and
a rotatable ring connecting to a proximal end of the first shaft and coaxially arranged with the first shaft;
a first and second control wires extended from the transmission unit,
connecting to the deflectable distal end respectively, wherein the first and second control wires are in contact with the rotatable ring and arranged to wrap around the first shaft in different directions;
wherein the rotatable ring is configured to apply a tensioning force to either the first or second control wire by rotation.

2. The cable-driven bending apparatus as claim 1, wherein the rotation of the rotatable ring simultaneously pulls one of the control wires and releases the other, thereby causing the deflectable distal end to bend.

3. The cable-driven bending apparatus as claim 1, wherein the first and second control wires are both secured to the rotatable ring.

4. The cable-driven bending apparatus as claim 3, wherein the first and second control wires are secured to a fixed point on the rotatable ring.

5. The cable-driven bending apparatus as claim 3, wherein the fixed points of the first and second control wires on the rotatable ring are different.

6. The cable-driven bending apparatus as claim 1, wherein, when the transmission unit is in a normal stage, the tensioning force on the first and second control wires is the same and the deflectable distal end is straight without any curvature, and both first and second control wire sections on the first shaft are equal in length, and the length satisfies the following equation:
$${L}_{NS} = \sqrt[2]{{H}^{2} + {( \frac{{θ}_{NS}}{180 °} \times π r )}^{2}} ,$$
wherein H represents a length of the first shaft, r represents an outer radius of the first shaft, π represents the mathematical constant pi, and θ_{NS} represents an angular difference between two ends of either control wire on the upper portion of the first shaft.

7. The cable-driven bending apparatus as claim 1, wherein, when the transmission unit is in a rotation stage, the tensioning force to the first and second control wires is different, and the deflectable distal end is bent and points to one side.

8. The cable-driven bending apparatus as claim 7, wherein both first and second control wire sections on the first shaft are not equal in length, and a length of the longer section satisfies the following equation:
$${L}_{RS} = \sqrt[2]{{H}^{2} + {( \frac{{θ}_{NS} + Δθ}{180 °} \times π r )}^{2}} ,$$
wherein H represents a length of the first shaft, r represents an outer radius of the first shaft, π represents the mathematical constant pi, θ_{NS} represents an angular difference between two ends of either control wire on the upper portion of the first shaft, and Δθ represents the rotation angle of the rotatable ring.

9. The cable-driven bending apparatus as claim 1, wherein the transmission unit further comprises a second shaft, and the rotatable ring is mounted between the first shaft and the second shaft.

10. The cable-driven bending apparatus as claim 9, wherein the first and second control wires are secured to the second shaft.

11. The cable-driven bending apparatus as claim 10, wherein the rotatable ring comprises a groove or a channel configured to accommodate the first and second control wires, and when the rotatable ring rotates, the first and second control wires are actuated such that one of the wires is pulled while the other is released.

12. The cable-driven bending apparatus as claim 1, wherein, when the transmission unit is in a rotation stage and the deflectable distal end reaches its maximum bending angle, one of the two control wires' sections on the first shaft has a shortest length.

13. The cable-driven bending apparatus as claim 12, wherein an extension direction of one of the two control wires' sections on the first shaft having the shortest length is along a direction of the transmission unit.

14. The cable-driven bending apparatus as claim 1, wherein the elongated body comprises a channel with an opening deposited at the proximal end of the elongated body, and the first and second control wires enter the channel through the opening and connect to the deflectable distal end.

15. The cable-driven bending apparatus as claim 1, wherein the elongated body comprises at least two channels and each of the channels has an opening deposited at the proximal end of the elongated body, and the first and second control wires each enter one of the channels through one of the openings and connect to the deflectable distal end.

16. A cable-driven bending apparatus, configured for removable coupling with a control, comprising:
an elongated body having a deflectable distal end and a proximal end;
a transmission unit positioned at a proximal end of the elongated body, comprising:
a first and second shafts extending along with the elongated body; and
a rotatable ring mounted coaxially between the first shaft and the second shaft;
a first and second control wires extended from the transmission unit,
connecting to the deflectable distal end respectively, wherein the other ends of the first and second control wires connect to the first shaft, and a portion of the first and second control wires are in contact with the rotatable ring and arranged to wrap around the first and second shafts in different directions;
wherein the rotatable ring is configured to apply a tensioning force on either the first or second control wire by rotation.

17. The cable-driven bending apparatus claim 16, wherein, when the transmission unit is in a rotation stage, the tensioning force on the first and second control wires is different, and the deflectable distal end is bent and points to one side.

18. A system, comprising:
the cable-driven bending apparatus as claim 1 or 22; and
a control handle, comprising:
a housing;
a controller installed on the housing; and
a coupling and actuation mechanism configured within the housing,
wherein the cable-driven bending apparatus is detachably connected with the control handle, and the controller drives and controls the transmission unit of the cable-driven bending apparatus via the coupling and drive mechanisms, thereby causing the deflectable distal end of the elongated body to bend.

19. The system as claim 18, wherein the cable-driven bending apparatus as claim 1 or 22 comprises a working channel having two openings, and the working channel extends through the elongated body and the transmission unit, wherein one opening is positioned at the deflectable distal end and the other opening is positioned at the proximal end of the transmission unit.

20. The system as claim 18, wherein the control handle comprises a working channel port detachably connected to the working channel.
